(19) United States
(12) Patent Application Publication
(10) Pub. No.: US 2012/0209010 A1
(43) Pub. Date: Aug. 16, 2012

(54) Method for the Manufacture of Fluorinated Ethylene Carbonates

(75) Inventors: Martin Bomkamp, Hannover (DE); Jens Olschimke, Hannover (DE); Johannes Eicher, Sehnde (DE)

(73) Assignee: SOLVAY FLUOR GMBH, Hannover (DE)

(21) Appl. No.: 13/502,583

(22) PCT Filed: Oct. 18, 2010
(86) PCT No.: PCT/EP10/65642

§ 371 Date: Apr. 18, 2012

(30) Foreign Application Priority Data
Oct. 21, 2009 (EP) 09173594.4

(51) Int. Cl. C07D 317/36; C25B 3/08

(52) U.S. Cl. 549/229; 205/427

(57) ABSTRACT

Difluoroethylene carbonate, trifluoroethylene carbonate and tetrafluoroethylene carbonate are synthesized from dichloroethylene carbonate, trichloroethylene carbonate and tetrachloroethylene carbonate with fluorinating agents, e.g. alkali metal fluorides, antimony fluorides and especially the HF adducts of amines The fluorinated carbonates are suitable as additives in lithium ion batteries.

### CROSS-REFERENCE TO RELATED APPLICATIONS

This application is a U.S. national stage entry under 35 U.S.C. §371 of International Application No. PCT/EP2010/065642 filed Oct. 18, 2010, which claims priority benefit to European Patent Application No. 09173594.4 filed on Oct. 21, 2009, the whole content of this application being incorporated herein by reference for all purposes.

### TECHNICAL FIELD OF THE INVENTION

The invention concerns a method for the manufacture of difluoroethylene carbonate, trifluoroethylene carbonate and tetrafluoroethylene carbonate by halogen-fluorine exchange, preferably chlorine-fluorine exchange.

### BACKGROUND OF THE INVENTION

International patent application WO 2009/107449 describes the manufacture of fluorinated carbonates by the reaction of halogenated organic carbonates with hydrofluoric acid addition salts of an amine in an organic solvent.

### SUMMARY OF THE INVENTION

Problem of the invention is to provide a process for the manufacture of difluoroethylene carbonate, trifluoroethylene and tetrafluoroethylene carbonate.

The problem is solved by the process as outlined in the claims.

According to the process of the present invention, difluoroethylene carbonate, trifluoroethylene carbonate and tetrafluoroethylene carbonate are manufactured from dichloroethylene carbonates, trichloroethylene carbonate and tetrachloroethylene carbonate by a halogen-fluorine exchange reaction with a fluorinating agent.

### DETAILED DESCRIPTION

Preferably, according to the process of the present invention, difluoroethylene carbonate, trifluoroethylene carbonate and tetrafluoroethylene carbonate are manufactured from trichloroethylene carbonate and tetrachloroethylene carbonate by a chlorine-fluorine exchange reaction with a fluorinating agent.

The term “halogen” denotes in the present invention chlorine, bromine and iodine. Preferably, the term “halogen” denotes chlorine.

Bromine-substituted ethylene carbonates and iodine-substituted ethylene carbonates can be provided by bromination or iodination of ethylene carbonate. These reactions can be supported photochemically. Iodine-substituted ethylene carbonate can also be manufactured by reacting ethylene carbonate and N-iodo compounds, e.g., N-iodo-acetamide or N-iodo-succinimide.

The invention will be explained in more detail in view of the chlorine-fluorine exchange which is by far the preferred embodiment.

Dichloroethylene carbonate can be manufactured from ethylene carbonate, and monochloroethylene carbonate by thermal or photochemical chlorination. If it is intended to manufacture predominantly dichloroethylene carbonate, the molar ratio of chlorine and each hydrogen atom to be substituted is preferably in the range of (0.8:1 to 1.2:1) multiplied by the number of hydrogen atoms to be substituted. Thus, if ethylene carbonate is applied as starting material, the ratio of chlorine to ethylene carbonate is preferably in the range of 1.6:1 to 2.4:1. If monochloroethylene carbonate is applied as starting material, the molar ratio of chlorine and monochloroethylene carbonate is preferably in the range of 0.8:1 to 1.2:1. The preferred ratio of chlorine and any mixtures of chlorinated carbonates can be calculated easily.

Trichloroethylene carbonate and tetrachloroethylene carbonate can be manufactured from ethylene carbonate, monochloroethylene carbonate and dichloroethylene carbonate (cis-4,5-trichloroethylene carbonate, trans-4,5-dichloroethylene carbonate, 4,4-dichloroethylene carbonate and any mixtures thereof) by thermal or photochemical chlorination. It has to be noted that in this reaction, trichloroethylene carbonate and tetrachloroethylene carbonate usually are produced simultaneously because trichloroethylene carbonate competes with lower chlorinated ethylene carbonates in the chlorination reaction. If it is intended to manufacture predominantly trichloroethylene carbonate, the molar ratio of chlorine and each hydrogen atom to be substituted is preferably in the range of (0.8:1 to 1.2:1) multiplied by the number of hydrogen atoms to be substituted. Thus, if ethylene carbonate is applied as starting material, the ratio of chlorine to ethylene carbonate is preferably in the range of 2.4:1 to 3.6:1. If monochloroethylene carbonate is applied as starting material, the molar ratio of chlorine and monochloroethylene carbonate is preferably in the range of 1.6:1 to 2.4:1. If dichloroethylene carbonate (any isomer or mixtures thereof can be used) is applied as starting material, the molar ratio between chlorine and dichloroethylene carbonate is preferably 0.8:1 to 1.2:1. The preferred ratio of chlorine and any mixtures of chlorinated carbonates can be calculated easily.

If predominantly tetrachloroethylene carbonate shall be manufactured, the preferred ratio between chlorine and any hydrogen atom present in the starting material is 0.9:1 to 1.1:1. Thus, if ethylene carbonate is applied as starting material, the ratio of chlorine to ethylene carbonate is preferably in the range of 3.6:1 to 4.4:1. If monochloroethylene carbonate is applied as starting material, the molar ratio of chlorine and monochloroethylene carbonate is preferably in the range of 2.7:1 to 3.3:1. If dichloroethylene carbonate (any isomer or mixtures thereof can be used) is applied as starting material, the molar ratio between chlorine and dichloroethylene carbonate is preferably 1.8:1 to 2.2:1. If trichloroethylene carbonate is applied as starting material, the molar ratio between chlorine and trichloroethylene carbonate is preferably 0.9:1 to 1.1:1. Also here, the preferred ratio of chlorine and any mixtures of chlorinated carbonates can be calculated easily on the basis of the explanation here above.

The manufacture of trichloroethylene carbonate and tetrachloroethylene carbonate by photochemical chlorination is preferred.

This embodiment will now be explained in further detail in view of the preferred starting material which is ethylene carbonate. Ethylene carbonate is kept in the liquid phase by warming it to 40° C. or more, or by adding a solvent, e.g., by adding chloroethylene carbonate or a fluorosubstituted ethylene carbonate. Alternatively or additionally, another solvent could be added, e.g., HF or a perfluorinated carbon compound. Chlorine gas, optionally diluted with inert gas, e.g., nitrogen, is introduced in gaseous form into the liquid under irradiation with UV light. If desired, an additional inert gas stream can be introduced into the photo reactor as described in US 20090082586.

If desired, the ethylene carbonate can be diluted with a suitable solvent, for example, chlorinated hydrocarbons, e.g., CCl₄, CH₂Cl₂, CHCl₃ or tetrachloroethane. Preferably, the reaction is performed in the absence of a solvent. The source for UV light is not critical. For example, high-pressure mercury lamps can be applied, as well as UV light emitting LEDs as described in WO 2009/013198. The light source can be separated from the reaction mixture by quartz glass or borosilicate glass. If desired, the glass surface can be protected by a shrinking wrap made from a polyfluorinated or perfluorinated polymer.

It may be advantageous to contact the reaction mixture with inert gas, e.g., nitrogen as described in US 2009/0082586, for example, by circulating a part of the reaction mixture through a loop reactor containing means to increase the contact surface. Such means are for example random packings or structured packings

The temperature of the liquid phase in the reactor is preferably kept in the range of 20 to 140° C.; if ethylene carbonate is applied as starting material, and no solvent, for example, chloroethylene carbonate or a fluorosubstituted ethylene carbonate, is used, then the temperature at the start of the reaction should be at least about 45° C., i.e. above the melting point of the ethylene carbonate. In early stages of the chlorination, a moderate temperature is recommended, e.g., up to 80° C. In later stages, the reaction can be performed in the upper temperature range, e.g., in a range of 80 to 140° C.

Specifically, the trichloroethylene carbonate can be manufactured as described in U.S. Pat. No. 4,535,175. After purging with nitrogen, ethylene carbonate is contacted with dry chlorine gas under irradiation with a lamp. The speed of chlorine introduction is regulated such that the liquid maintains a slightly yellow color. The initial temperature was 35° C., and up to 115° C. during the later part of the reaction. The reaction is terminated when all 4-chloroethylene carbonate is consumed (as indicated by gas chromatographic analysis). The product is principally trichloroethylene carbonate with lesser amounts of dichloroethylene carbonate and tetrachloroethylene carbonate.

Tetrachloroethylene carbonate can be produced as described in EP-A-0080187. A reactor is charged with molten ethylene carbonate, and after purging with nitrogen, chlorine is introduced such that the liquid remains yellow. The initial reaction temperature is kept lower than 80° C. in the first hours of chlorination; later, it can be increased to 100 to 120° C. The chlorination is continued until gas chromatographic analysis reveals that incompletely chlorinated intermediates are absent.

As mentioned above, the reaction mixture often contains trichloroethylene carbonate and tetrachloroethylene carbonate, possibly also dichloroethylene carbonate. If desired, trichloroethylene carbonate and tetrachloroethylene carbonate can be isolated, especially by distillation. For further reaction of the chlorocarbonates to form fluorocarbonates, isolation of the intermediates is not necessary. The boiling point of tetrachloroethylene carbonate is 46° C. at 666 Pa.

It is preferred to remove a part or even most of the HCl which is a reaction product. This can be done by stripping the HCl from the reaction mixture, e.g., by passing inert gas, especially hot inert gas through it. A stripping column is highly suitable in which the reaction mixture is introduced at or near the top of the column, and the stripping gas is introduced at the bottom or near the bottom of the column.

The fluorination reaction is performed in a common reactor. Stirred reactors are very suitable. Since the reaction products difluoroethylene carbonate, trifluoroethylene carbonate and tetrafluoroethylene carbonate are sensitive towards hydrolysis, water is excluded. For example, the reactor can be purged with dry inert gas, especially nitrogen, before the reaction is performed.

Generally, any agent known to be suitable for chlorine-fluorine exchange reactions can be applied. For example, metal fluorides or their HF adducts can be applied. Suitable are, for example, alkali metal fluorides and their HF adducts of general formula MF.nHF wherein M is the cation of an alkali metal, and n is 1, 2, 3 or even greater. Of this type of compounds, KF, KF.HF, CsF, CsF.HF, KF.2HF and CsF.2HF are especially preferred. Alkaline earth metal fluorides and group V metal fluorides, as well as transition metal fluorides and any HF adducts thereof, are also applicable. Very suitable compounds of this type are, for example, CaF₂, SbF₃, SbF₅, AsF₃, AsF₅, and AgF. If desired, a solvent, with the exception of water, can be applied. COF₂ is also suitable as fluorinating agent. Ammonium fluoride and amine fluorides and their HF adducts are also suitable, for example, NH₃.nHF wherein n is 1 to 10, preferably, 1 to 3.

It is preferred to apply HF adducts of amines. HF adducts of amines preferably are those of formula (I), R¹R²R³N.nHF. Here, n is 1 to 10, preferably, 1 to 4. More preferably, n is equal to or greater than 1 and equal to or lower than 4. At least one of R¹, R², and R³ is an organic group, especially, an alkyl group, a phenyl group, a benzyl group, or 2 or all 3 groups R′, R², and R³ form a 4-membered to 7-membered ring which includes the nitrogen atom.

Preferably, in the HF adducts of amines of formula (I), R¹, R², and R³ are the same or different and denote H, alkyl with 1 to 10 carbon atoms, phenyl or benzyl; alternatively, 2 substituents of R¹, R², and R³ or all three substituents form a ring which includes the nitrogen atom; at least one of R¹, R², and R³ is not hydrogen, but one of said organic groups. Optionally, R¹ and R² form a ring which includes the nitrogen atom; here, the ring preferably is a 4-membered ring, a 5-membered ring or a 6-membered ring. It may be a saturated or unsaturated ring. It can contain carbon atoms or further hetero atoms, for example, it may contain in total 2 or 3 N atoms. For example, the HF adducts of pyridine, aniline, or chinoline are suitable as fluorinating agents.

Preferably, in the formula (I), R¹, R², and R³ are the same or different and denote H, alkyl with 1 to 4 carbon atoms with the proviso that at least one of R¹, R², and R³ is alkyl with 1 to 4 carbons, and n is equal to or greater than 1 and equal to or lower than 4. More preferably, R¹, R², and R³ are the same or different and denote methyl, ethyl, n-propyl or isopropyl. Especially preferably, R¹, R², and R³ are the same and denote methyl, ethyl, n-propyl or isopropyl.

Most preferably, n is equal to or greater than 2.5.

Most preferably, n is equal to or lower than 3.5.

The HF adducts of triethyl amine are especially suitable, especially those with the formula Et₃N.nHF wherein Et denotes the ethyl group and n is 2.5 to 3.5. They are liquid at ambient temperature and can easily be purified by distillation.

It is known that the HF content of the HF adducts reacts with the chlorinated ethylene compounds under formation of the fluorinated ethylene carbonates and HCl. It is assumed that further HF is liberated from the HF adducts when n is greater than 1 when a part of the HF from the HF adduct is consumed in the fluorination reaction. Thus, according to one preferred embodiment, when HF adducts of amines with a fluorine/amine ratio greater than 1 are applied, i.e., in the compound of formula (I), n is greater than 1, simultaneously to the course of the reaction, the respective free amine is added. It is assumed that any liberated HF is captured by the added amine, and is utilized for chlorine-fluorine exchange. The addition of amine prevents that HF leaves the reaction mixture as vapor.

The amount of the amine added corresponds to (n−1) of the respective value of n in the HF adduct of the amine with the general formula R¹R²R³N.nHF. Thus, if for example, 1 mol of Et₃N.3HF is applied which is the most preferred compound of formula (I), (3−2) moles of Et₃N are added.

This embodiment is especially preferred if amine adducts of formula (I) are applied wherein n is equal to or greater than 2.5 and equal to or lower than 3.5.

If the reaction is performed under pressure which retains any gaseous constituents in the reaction mixture, for example, in a batch reactor under autogenous pressure or applying a cooler in which HF leaving the reaction mixture as vapor is condensed and returned to the reaction mixture, it is not necessary to add amine.

Most of the HF adducts of the amines are liquid. Thus, it is a priori not necessary to apply a solvent.

If desired, a solvent, with the exception of water, can be applied. Preferred solvents are aprotic organic solvents. For example, ethers, e.g., diethyl ether, ketones, for example, acetone or butyl methyl ketone, halogenated hydrocarbons, for example, dichloromethane, chloroform, tetrachloromethane, tetrachloroethane, nitriles, for example, acetonitrile or adiponitrile, or amides, e.g., formamide, ethers, for example, ethers of glycols or polyglycols, for example, diglyme or triglyme, can be applied as solvent.

If a solvent is present, its content in the reaction mixture is preferably in a range of 10 to 80% by weight, the total amount of the reaction mixture set to 100% by weight.

The temperature of the fluorination mixture is selected such that the fluorination takes place in a reasonable reaction rate. The reaction is preferably performed at a temperature equal to or greater than 60° C. Preferably, the reaction temperature is equal to or lower than 200° C.

The pressure can vary in a broad range. If the reaction is performed using an HF adduct of an amine, HCl is a reaction product. If the reaction is performed in an open system, and if gaseous products, mainly HCl, are purged, then it is advisable to be aware that difluoroethylene carbonate, trifluoroethylene carbonate and tetrafluoroethylene carbonate have a rather low boiling point and may leave the reactor entrained in the gaseous products. The gases leaving the reactor should be passed through a cooled trap or traps, or a cooler should be applied to condense organic components from the gas leaving the reactor. Any difluoroethylene carbonate, trifluoroethylene carbonate or tetrafluoroethylene carbonate can be separated by distillation or fractionated condensation, precipitation or crystallization; or they are returned to the reaction mixture. A pressure in the range of 1 to 30 bar (abs.) is preferred. The pressure should be at least so high that the organic constituents remain essentially in the liquid phase.

The reaction can also be performed in an autoclave. This has the advantage that no loss of low-boiling product occurs during the reaction.

The molar ratio of fluoride in the metal fluoride or the HF adduct and chlorine atoms to be substituted in the chlorosubstituted ethylene carbonate is preferably equal to or greater than 1:1. It can be lower than 1:1, but then the yield deteriorates. Preferably, the ratio is equal to or lower than 2:1. It may be higher, e.g., up to 3:1, but, unless recovered or used for subsequent fluorination batches, a part of the fluorinated reactant might be wasted.

The raw product contains difluoroethylene carbonate, trifluoroethylene carbonate and/or tetrafluoroethylene carbonate, possibly chlorofluorinated intermediates and/or unreacted starting material, metal chloride or amine hydrochloride and possibly HCl and/or HF. Difluoroethylene carbonate, trifluoroethylene carbonate and tetrafluoroethylene carbonate can be isolated in a manner generally known. It is preferred not to perform an aqueous workup of the reaction mixture. HCl can be removed from the reaction mixture by vaporizing the reaction mixture and passing the vapors through cooled traps which let HCl pass, while components, especially difluoroethylene carbonate, trifluoroethylene carbonate and/or tetrafluoroethylene carbonate, are condensed. Traps cooled to 0° C. to −180° C. are highly suitable. HF can be removed by passing the reaction mixture or raw distillates over a metal fluoride, especially sodium fluoride or potassium fluoride. The reaction mixture or the pre-purified raw product from the traps and/or HF removal can be subjected to a fractionated condensation. The separation of difluoroethylene carbonate, trifluoroethylene carbonate and/or tetrafluoroethylene carbonate from the reaction mixture or any fraction thereof is possible by distillation, especially pressure distillation or deep temperature distillation.

If HF additives of amines are applied the respective hydrochlorides of the amines are formed as reaction products. These amines can be regenerated by passing HF through them. Difluoroethylene carbonate, trifluoroethylene carbonate or tetrafluoroethylene carbonate are suitable as solvent, as etching agent, as fire extinguishing agent and especially as solvent or solvent additive for Li ion batteries.

Isolated trichloroethylene carbonate is novel and also subject of the present invention. This compound can be isolated from the reaction mixture obtained by chlorination as described further above. Trichloroethylene carbonate is a precursor of trifluoroethylene carbonate and, after further chlorination, of tetrafluoroethylene carbonate which are valuable compounds, e.g., as solvents or solvent additives for Li ion batteries.

The advantage of the present process is that difluoroethylene carbonate, trifluoroethylene carbonate and tetrafluoroethylene carbonate can be manufactured in a technically simple manner.

Difluoroethylene carbonate, trifluoroethylene carbonate and tetrafluoroethylene carbonate can also be manufactured from ethylene carbonate by electro fluorination. Ethylene carbonate or fluorosubstituted ethylene carbonate precursors, for example, any of the isomers of difluoroethylene carbonate, or, for the manufacture of tetrafluoroethylene carbonate, also trifluoroethylene carbonate, are electro fluorinated in liquid HF at cell voltages of 5 to 6 V. The concentration of the starting material in HF should be in the range of 1 to 50% by weight. Preferably, the concentration of the starting material in HF is equal to or lower than 15% by weight. A good stirring is recommended. The current density is adjusted such that optimal yields are obtained. A current density of 20 to 50 mA/cm² gives good yields. It is advisable to add further HF and further starting material at the same rate of the fluorination reaction. A suitable apparatus comprising a 900 ml stainless steel cylindrical cell with a pack of alternate nickel cathodes and anodes and an effective anodes area of 630 cm² is described in “3. Experimental details” on page 142 of the publication of Lino Conte and GianPaolo Gambaretto in J. Fluorine Chem. 125 (2004), 139-144. The cell was provided with a liquid level indicator and with a condenser maintained at −40° C. to condense hydrogen fluoride and entrained fluorosubstituted organic products in the gas stream exiting from the cell. Consequently, the process for the manufacture of difluoroethylene carbonate, trifluoroethylene carbonate and tetrafluoroethylene carbonate from ethylene carbonate or fluorosubstituted ethylene carbonate precursors by electro fluorination in liquid HF is another aspect of the present invention. The cell voltage is preferably in the range of 5 to 6 V. The concentration of the precursor is kept preferably in a range of 5 to 15% by weight in HF solution.

After the termination of the reaction, HF is removed for example by adding an HF scavenger, e.g., NaF. The remaining organic crude product can be separated by distillation to obtain the desired difluoroethylene carbonate, trifluoroethylene carbonate and tetrafluoroethylene carbonate.

The following examples are intended to explain the reaction in further detail without intending to limit. Should the disclosure of any patents, patent applications, and publications which are incorporated herein by reference conflict with the description of the present application to the extent that it may render a term unclear, the present description shall take precedence.

### EXAMPLES

### Example 1

### Manufacture of Trichloroethylene Carbonate and Tetrachloroethylene Carbonate

The reaction principally is performed as described in [0042] of US 2009/0082586.

The apparatus comprises a photochemical reactor, a receiving vessel and a packed column. Ethylene carbonate is put in the receiving vessel, heated until it melts, and then it is pumped through the photo reactor. Chlorine gas is introduced into the photo reactor and contacted therein with liquid phase. The photo reactor comprises a UV light lamp, for example, a 150 W high pressure mercury lamp of Heraeus. Chlorine gas is fed continuously into the liquid phase at the bottom of the reactor. 800 l/h of nitrogen gas are fed to the liquid phase at the top area of the irradiation reactor and into the liquid phase at the bottom of the receiving vessel. The temperature of the liquid phase in the irradiation reactor is initially kept in a range of 40 to 45° C., later up to 80° C. Finally, the temperature of the reaction mixture is kept at about 120° C. The liquid phase is pumped to circulate with a flow rate in the range from 2 to 3 l/min. After 18 kg of chlorine gas are introduced, the reaction is stopped. The liquid phase contains essentially only trichloroethylene carbonate and tetrachloroethylene carbonate.

### Example 2

### Fluorination of Trichloroethylene Carbonate and Tetrachloroethylene Carbonate to Trifluoroethylene Carbonate and Tetrafluoroethylene Carbonate with KF.HF

33% by weight of the liquid reaction mixture of example 1 is transferred without further purification into a stirred autoclave serving as a fluorination reactor. Dry acetonitrile is added such that the concentration of acetonitrile in the resulting liquid phase is about 50% by volume.

KF.HF is added such that about 0.5 molecules of KF.HF is applied per chlorine atom to be substituted. The autoclave is closed, stirring is started, and the reaction mixture is heated to about 120° C. Samples are taken from the reaction mixture, and the conversion of trichloroethylene carbonate and tetrachloroethylene carbonate to trifluoroethylene carbonate and tetrafluoroethylene carbonate are controlled. The reaction is continued until the desired degree of conversion is achieved.

Solids are removed from the reaction mixture by filtration, and the resulting filtrate is distilled under pressure. The boiling point of tetrafluoroethylene carbonate is about 46° C. at a pressure of 666 Pa.

### Example 3

### Fluorination of Trichloroethylene Carbonate and Tetrachloroethylene Carbonate with SbF₃

33% by weight of the liquid reaction mixture of example 1 is transferred without further purification into a stirred autoclave serving as a fluorination reactor. Dry acetonitrile is added such that the concentration of acetonitrile in the resulting liquid phase is about 50% by volume.

Freshly dried SbF₃ is added such that about 0.4 molecules of SbF₃ are applied per chlorine atom to be substituted. The autoclave is closed, stirring is started, and the reaction mixture is heated to about 150° C. Samples are taken from the reaction mixture, and the conversion of trichloroethylene carbonate and tetrachloroethylene carbonate to trifluoroethylene carbonate and tetrafluoroethylene carbonate are controlled. The reaction is continued until the desired degree of conversion is achieved.

The resulting reaction mixture is distilled under pressure. SbCl₃ has a melting point of about 74° C. and a boiling point of about 223° C. and can easily be separated from the fluorinated reaction products.

### Example 4

### Fluorination of Trichloroethylene Carbonate and Tetrachloroethylene Carbonate with Et₃N.3HF

33% by weight of the liquid reaction mixture of example 1 is transferred without further purification into a stirred autoclave serving as a fluorination reactor. No solvent is added.

The reaction is performed in a reactor with attached condenser. The condenser is cooled to −58° C. to condense any gaseous or vaporous compounds which then are returned to the reactor.

Freshly distilled Et₃N.3HF (available from Sigma-Aldrich or by the reaction of HF and Et₃N in a molar ratio of 3:1; boiling point: 77° C. at 15 mm Hg) is slowly added. The reaction mixture is kept in a range of 80 to 100° C. When the reaction starts, hydrochloride is formed. Also, HF is liberated. To bind this liberated HF, triethylamine is slowly added, too, to the reaction mixture. The total amount of the Et₃N.3HF is selected such that about 0.4 molecules of Et₃N.3HF are added per molecule of chlorine to be substituted. The molar ratio of triethylamine and Et₃N.3HF is about 2:1. Samples are taken from the reaction mixture, and the conversion of trichloroethylene carbonate and tetrachloroethylene carbonate to trifluoroethylene carbonate and tetrafluoroethylene carbonate are controlled. The reaction is continued until the desired degree of conversion is achieved.

The resulting reaction mixture is distilled under pressure. Any solids can be removed beforehand by filtration.

### Example 5

### Manufacture of Trifluoroethylene Carbonate by Electro Fluorination

In a reactor as described by Conte and Gambaretto, vide supra, with a volume of 900 ml, and nickel electrodes, ethylene carbonate (EC) is dissolved in dry HF to provide a solution containing 12% by weight of EC. The cell is provided with a condenser maintained at −40° C. The voltage is kept between 5.4 and 5.7 V. According to the liquid level indicator, HF and EC were introduced into the reactor during the course of the reaction. Regularly, samples are taken from the reaction mixture and analyzed.

The addition of EC and HF is stopped, and after the conversion of EC has reached the desired level, the reaction mixture is contacted with an adsorbent for HF, e.g., NaF. The crude organic phase is separated from the solid salt and distilled under pressure to isolate pure tetrafluoroethylene carbonate.

### Example 6

### Manufacture of Trans-Difluoroethylene Carbonate in Acetonitrile

In a 250 mL perfluoroalkoxyethylene (PFA) flask connected to a reflux condenser 15 g of trans-4,5-dichloro-1,3-dioxolan-2-one (dichloroethylene carbonate) were dissolved in 100 mL dried acetonitrile. After addition of 22 g of potassium fluoride the mixture was stirred under reflux for 18 h. The reaction mixture was allowed to cool to room temperature before the insoluble parts were removed by filtration and the filter cake was washed with 20 mL of acetonitrile. The product (trans-difluoroethylene carbonate, “trans-F2EC”) was isolated by distillation. The product was obtained as a colorless liquid (6.2 g).

### Example 7

### Manufacture of Trans-Difluoroethylene Carbonate in Methyl-Tert-Butyl Ether

To a solution of 5 g trans-4,5-dichloro-1,3-dioxolan-2-one in 50 mL methyl-tert-butyl ether in a 250 mL PFA-flask 10 g of 1,5-diazabicyclo[4.3.0]non-5-ene(DBN).2.6 HF were added. After vigorous stirring of for 48 h at room temperature trans-F2EC could be proven by gas chromatographic (GC) and gas chromatography-mass spectrometry (GCMS).

1. A method for the manufacture of difluoroethylene carbonate, trifluoroethylene carbonate or tetrafluoroethylene carbonate from dichloroethylene carbonate, trichloroethylene carbonate or tetrachloroethylene carbonate, respectively, by a chlorine-fluorine exchange reaction with a fluorinating agent, or by electro fluorination of ethylene carbonate or a fluorosubstituted ethylene carbonate precursor with lower degree of fluorination.

2. The method of claim 1 for the manufacture of difluoroethylene carbonate, trifluoroethylene carbonate or tetrafluoroethylene carbonate from dichloroethylene carbonate, trichloroethylene carbonate or tetrachloroethylene carbonate, respectively, by the chlorine-fluorine exchange reaction with a fluorinating agent.

3. The method of claim 2 wherein the fluorinating agent is selected from the group consisting of metal fluorides, ammonium fluoride, amine fluoride, and HF adducts thereof.

4. The method of claim 2 wherein the fluorinating agent is an HF adduct of an alkali metal fluoride.

5. The method of claim 4 wherein the fluorinating agent is selected from the group consisting of KF.HF, CsF.HF, KF.2HF and CsF.2HF.

6. The method of claim 3 wherein the fluorinating agent is a metal fluoride selected from the group consisting of SbF₃, SbF₅ and their HF adducts.

7. The method of claim 3 wherein the fluorinating agent is selected from the group consisting of HF adducts of amines with a general formula (I): R¹R²R³N.nHF, wherein n is 1 to 10, and wherein R¹, R², and R³ are the same or different and are selected from the group consisting of H, alkyl with 1 to 10 carbon atoms, phenyl, and benzyl; or wherein two of the substituents R¹, R², and R³ or all three substituents R¹, R², and R³ form a ring which includes the nitrogen atom.

8. The method of claim 7 wherein n is from 1 to 4.

9. The method of claim 7 wherein R¹, R², and R³ are the same or different and are selected from the group consisting of methyl, ethyl, n-propyl, and isopropyl.

10. The method of claim 9 wherein the fluorinating agent is triethylamine tris-hydro fluoride.

11. The method of claim 7 wherein an amine of formula R¹R²R³N is simultaneously applied wherein R¹, R², and R³ in formula: R¹R²R³N are the same as in the general formula (I): R¹R²R³N.nHF, and wherein the amine of formula R¹R²R³N corresponds to the amine group in the HF adducts of amines with the general formula (I) R¹R²R³N.nHF with the proviso that n is greater than 1.

12. The method of claim 11 wherein the amount of said amine of formula R¹R²R³N added corresponds to (n−1) of the respective value of n in the HF adduct of the amine with the general formula R¹R²R³N.nHF.

13. The method of claim 1 wherein trichloroethylene carbonate or tetrachloroethylene carbonate is produced by photo-induced liquid phase reaction of ethylene carbonate, monochloroethylene carbonate, dichloroethylene carbonate, or any mixtures thereof, with chlorine.

14. The method of claim 1 wherein trifluoroethylene carbonate or tetrafluoroethylene carbonate is manufactured.

15. Isolated trichloroethylene carbonate.

16. The method of claim 1 wherein ethylene carbonate or fluorinated ethylene carbonate precursors are subjected to electro fluorination in liquid HF.

17. The method of claim 16 wherein a cell voltage of from 5 to 6 V is applied.

18. The method of claim 16 wherein a current density of from 20 to 50 mA/cm² is applied.

19. The method of claim 16 being performed in a cell which comprises a liquid level indicator and a condenser.

20. The method of claim 16 wherein the concentration of the starting material in HF is equal to or lower than 15% by weight.
